(19) United States
(12) Patent Application Publication
(10) Pub. No.: US 2017/0106011 A1
(43) Pub. Date: Apr. 20, 2017

(54) METHODS OF TREATING TRAUMATIC BRAIN INJURY AND SEQUELAE

(71) Applicant: Cantex Pharmaceuticals, Inc., Weston, FL (US)

(72) Inventor: Stephen Marcus, Weston, FL (US)

(21) Appl. No.: 15/294,689

(22) Filed: Oct. 15, 2016

Related U.S. Application Data
(60) Provisional application No. 62/244,125, filed on Oct. 20, 2015.

(51) Int. Cl. A61K 31/727; A61K 9/00

(52) U.S. Cl. CPC A61K 31/727; A61K 9/0019

(57) ABSTRACT

Methods and compositions are presented for treating traumatic brain injury and complications of traumatic brain injury, including pulmonary complications and cerebral edema.

### CROSS-REFERENCE TO RELATED APPLICATIONS

This application claims the benefit under 35 USC 119(e) of prior co-pending U.S. Provisional Patent Application No. 62/244,125, filed Oct. 20, 2015, the disclosure of which is hereby incorporated by reference in its entirety.

### 1. BACKGROUND

Approximately 92,000 people in the US are hospitalized each year with moderate to severe traumatic brain injury. Virtually all survivors of moderate or severe TBI develop significant long-term neurobehavioral sequelae. Ongoing “secondary” injury after initial injury plays a major role in worsening outcome. Economic costs associated with traumatic brain injury are enormous. There are currently no drugs available to prevent the ongoing “secondary” brain damage after initial injury. There is a need in the art for therapeutic agents to treat primary and secondary traumatic brain injury and complications of brain injury.

### 2. SUMMARY

In a first aspect, methods of treatment are provided.

### 3. BRIEF DESCRIPTION OF THE DRAWINGS

FIG. 1 outlines primary and secondary traumatic brain injury (TBI) symptoms.

FIG. 2A shows the chemical formula of the ATIII-binding pentasaccharide sequence of unfractionated heparin (“UFH”) and the comparable sequence of 2-O, 3-O-desulfated heparin (“ODSH”) prepared by cold alkaline hydrolysis of UFH.

FIG. 2B shows the repeating disaccharide unit of ODSH.

FIGS. 3A-3C show reduction of secondary brain injury with ODSH (CX-01) after middle cerebral artery occlusion in an animal model.

FIG. 4 outlines the phase II clinical trial of ODSH for treating TBI.

### 4. DETAILED DESCRIPTION

### 4.1. Methods of Treating

In one aspect, methods of treating are provided for treating a subject suffering from a disorder selected from the group consisting of: traumatic brain injury (TBI), lung injury associated with TBI, traumatic spine injury, intracerebral hemorrhage, subarachnoid hemorrhage, and acute respiratory distress syndrome (ARDS). The methods comprise administering to the subject an effective amount of non-anticoagulating, non-LMWH heparinoid. Non-anticoagulating, non-LMWH heparinoids for use in the methods are described in Section 4.2.

### 4.1.1. Methods of Treating TBI

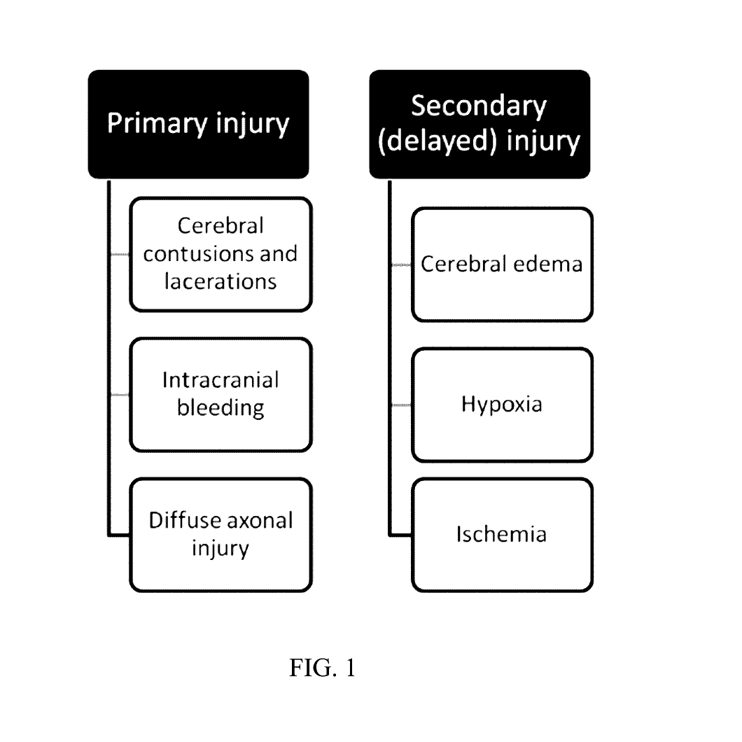

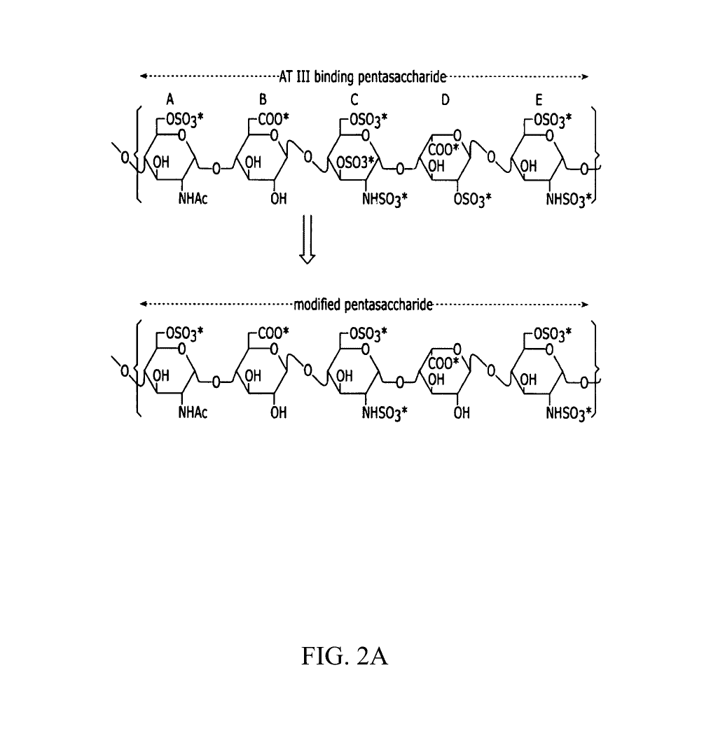

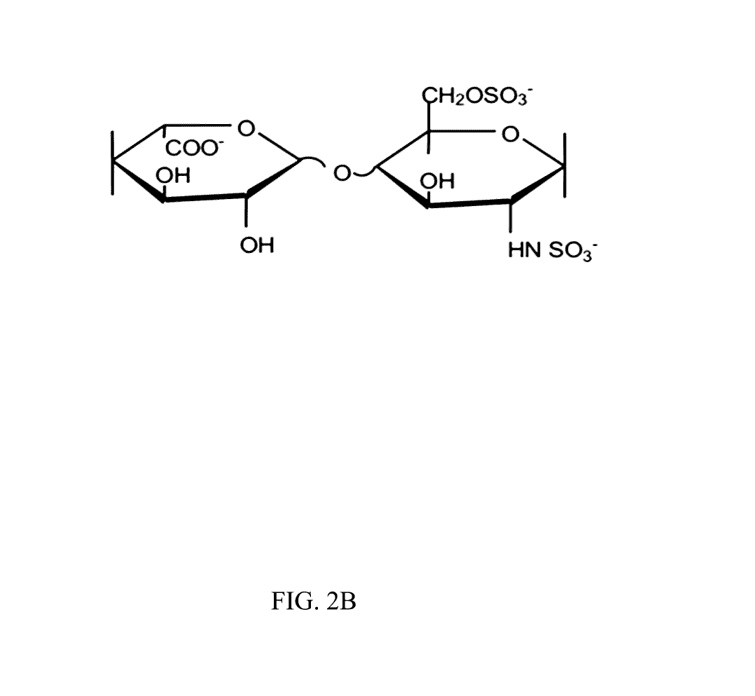

In certain embodiments, methods are provided for treating a subject suffering from traumatic brain injury (TBI). FIG. 1 outlines primary and secondary traumatic brain injury (TBI) symptoms. In these embodiments, the non-anticoagulating, non-LMWH heparinoid is administered as soon following brain injury as possible.

### 4.1.2. Methods of Treating Lung Injury Associated with TBI

In certain embodiments, methods are provided for treating a subject suffering from lung injury following TBI. In some embodiments, the lungs are subsequently transplanted from the treated donor into a recipient. In certain of these embodiments, the recipient is also treated.

### 4.1.3. Methods of Treating Acute Spinal Injury

In certain embodiments, methods are provided for treating a subject suffering from acute injury to the spine, including injury to the spinal cord. In these embodiments, the non-anticoagulating, non-LMWH heparinoid is administered as soon following brain injury as possible.

### 4.1.4. Methods for Treating Intracerebral Hemorrhage

In some embodiments, methods are provided for treating acute respiratory distress syndrome (ARDS

### 4.1.5. Methods for Treating Subarachnoid Hemorrhage

In some embodiments, methods are provided for treating subarachnoid hemorrhage.

### 4.1.6. Methods for Treating ARDS

In some embodiments, methods are provided for treating acute respiratory distress syndrome (ARDS).

### 4.2. Effective Heparins and Heparin Derivatives

In the methods described herein, the heparin or heparin derivative is a substantially non-anticoagulating heparin derivative with sufficient negative charge density to bind to proteins having regions of net positive charge. For convenience, such heparins and heparin derivatives are collectively referred to herein as “non-anticoagulating , non-LMWH, heparinoids”.

In certain preferred embodiments, the non-anticoagulating, non-LMWH, heparinoid is a derivative of unfractionated heparin that is substantially desulfated at the 2-O position of α-L-iduronic acid (referred to herein as the “2-O position”) and/or 3-O position of D-glucosamine-N-sulfate (6-sulfate) (referred to herein as the “3-O position”). In preferred embodiments, the 2-O, 3-O-desulfated heparin derivative is not substantially desulfated at the 6-O or N positions.

In some of these embodiments, the non-anticoagulating, non-LMWH, heparinoid is at least 85%, at least 90%, at least 95%, or at least 99% desulfated at the 2-O position. In some embodiments, the non-anticoagulating, non-LMWH, heparinoid is at least 85%, at least 90%, at least 95%, or at least 99% desulfated at the 3-O position. In some embodiments, the non-anticoagulating, non-LMWH, heparinoid is at least 85%, at least 90%, at least 95%, at least 99% desulfated at the 2-O position and the 3-O position.

In some of these embodiments, the non-anticoagulating, non-LMWH, heparinoid has an average molecular weight from about 2 kDa to about 15 kDa. In some embodiments, the non-anticoagulating, non-LMWH, heparinoid has an average molecular weight of at least about 2 kDa, at least about 3 kDa, at least about 4 kDa, at least about 5 kDa, at least about 6 kDa, or at least about 7 kDa. In some embodiments, the non-anticoagulating, non-LMWH, heparinoid has an average molecular weight of less than about 15 kDa, less than about 14 kDa, less than about 13 kDa, less than about 12 kDa, less than about 11 kDa, less than about 10 kDa, or less than about 9 kDa. In some embodiments, the average molecular weight of the non-anticoagulating, non-LMWH, heparinoid is selected from about 2 kDa, 3 kDa, 4 kDa, 5 kDa, 6 kDa, 7 kDa, 8 kDa, 9 kDa, 10 kDa, 11 kDa, 12 kDa, 13 kDa, 14 kDa, 15 kDa, 16 kDa, 17 kDa, 18 kDa, or a range that includes any of these values as endpoints.

In some embodiments, the substantially 2-O, 3-O desulfated non-anticoagulating, non-LMWH, heparinoid for use in the methods described herein is a compositions in which the polysaccharide chains have an average molecular weight of at least about 8 kDa. In some embodiments, the substantially 2-O, 3-O desulfated non-anticoagulating, non-LMWH, heparinoids have an average molecular weight of greater than about 8 kDa. In various embodiments, the substantially 2-O, 3-O desulfated non-anticoagulating, non-LMWH, heparinoids have an average molecular weight ranging from about 8 kDa to about 15 kDa. In some embodiments, the substantially 2-O, 3-O desulfated non-anticoagulating, non-LMWH, heparinoids for use in the methods described herein have an average molecular weight that ranges in size from about 11 kDa to about 13 kDa.

Molecular weight of heparinoids can be determined by methods know in the art; for purposes of this disclosure, molecular weight is determined by size exclusion chromatography as described in Lapierre et al., Glycobiology 6(3):355-366 (1996), incorporated herein by reference in its entirety.

An exemplary non-anticoagulating, non-LMWH, heparinoid for use in the methods described herein is substantially 2-O, 3-O desulfated heparin, referred to herein as ODSH. ODSH for use in the above-described methods can be prepared from bovine or porcine heparin. In an exemplary method of preparing ODSH from porcine heparin, ODSH is synthesized by cold alkaline hydrolysis of USP porcine intestinal heparin, which removes the 2-O and 3-O sulfates, leaving N- and 6-O sulfates on D-glucosamine sugars and carboxylates on αL-iduronic acid sugars substantially intact (Fryer et al., J Pharmacol. Exp. Ther. 282: 208-219 (1997), incorporated herein by reference in its entirety). Using this method, ODSH can be produced with an average molecular weight of about 11.7±0.3 kDa. Additional methods for the preparation of substantially 2-O, 3-O desulfated non-anticoagulating, non-LMWH, heparinoids may also be found, for example, in U.S. Pat. Nos. 5,668,118, 5,912,237, and 6,489,311, and WO 2009/015183, the contents of which are incorporated herein in their entirety, and in U.S. Pat. Nos. 5,296,471, 5,969,100, and 5,808,021.

In contrast to unfractionated heparin, ODSH is substantially non-anticoagulating: administered to a subject at a dose that is equivalent in weight to a fully-anticoagulating dose of unfractionated heparin, the clotting time measured in an aPTT assay is no greater than 45 seconds, and typically in the upper range of normal, where normal clotting time ranges from about 27 to 35 seconds. By comparison, unfractionated heparin administered to a subject at a fully anticoagulant dose causes time to clot to range from about 60 to about 85 seconds in an aPTT assay.

Thus, the non-anticoagulating, non-LMWH, heparinoid is substantially non-anticoagulating. In preferred embodiments, if the non-anticoagulating, non-LMWH, heparinoid is administered to subject at a dose that is weight equivalent to a fully-anticoagulating dose of unfractionated heparin, the subject's clotting time measured in an aPTT assay is no greater than 45 seconds.

Another measure of ODSH's anticoagulant activity is its anti-Xₐ activity which can be determined in an assay carried out using plasma treated with Russell viper venom. In specific examples, ODSH exhibited less than 9 U of anticoagulant activity/ mg in the USP anticoagulant assay (e.g., 7±0.3 U), less than 5 U of anti-Xₐ activity/mg (e.g., 1.9±0.1 U/mg) and less than 2 U of anti-IIₐ activity/mg (e.g., 1.2±0.1 U/mg) (compared to unfractionated heparin which has an activity of 165-190 U/mg in all three assays; Rao et al., Am. J. Physiol. 299:C97-C110 (2010)). Thus, in certain embodiments, the non-anticoagulating, non-LMWH, heparinoid exhibits less than 9 U of anticoagulant activity/mg in the USP anticoagulant assay, and/or less than 5 U of anti-Xₐ activity/mg, and/or less than 2 U of anti-IIₐ activity/mg.

Furthermore, ODSH has a low affinity for anti-thrombin III (Kd˜339 μM or 4 mg/ml vs. 1.56 μM or 22 μg/ml for unfractionated heparin), consistent with the observed low level of anticoagulant activity, measured as described in Rao et al., supra, at page C98. Thus, in certain embodiments, the non-anticoagulating, non-LMWH, heparinoid has a low affinity for anti-thrombin III (Kd˜339 μM or 4 mg/ml).

In some embodiments, the non-anticoagulating, non-LMWH, heparinoids have no more than 40% of the anticoagulating activity of an equal weight of unfractionated heparin by any one or more of the above-described tests. In some embodiments, the non-anticoagulating, non-LMWH, heparinoid has no more than 35%, no more than 30%, no more than 20%, no more than 10%, no more than 9%, no more than 8%, no more than 7%, no more than 6%, no more than 5%, no more than 4%, no more than 3%, no more than 2%, or no more than 1% of the anti-coagulating activity of an equal weight of unfractionated heparin by any one or more of the above-described tests.

In some embodiments, the non-anticoagulating, non-LMWH, heparinoid does not trigger platelet activation and does not induce heparin-induced thrombocytopenia (HIT). Platelet activation can be determined using a serotonin release assay, for example as described in U.S. Pat. No. 7,468,358 and Sheridan et al., Blood 67:27-30 (1986)), incorporated herein by reference. In some embodiments, the heparinoid is capable of binding platelet factor 4, also referred to as chemokine (C-X-C motif) ligand 4 (CXCL4).

### 4.2.1. Modes and Routes of Administration

The non-anticoagulating, non-LMWH, heparinoid can be administered in the methods by any one or more of a variety of routes.

In certain embodiments, the heparinoid is administered intravenously.

In certain intravenous embodiments, the heparinoid is administered by bolus intravenous administration. In some embodiments, a bolus dose is administered over less than a minute, about a minute, about 2 minutes, about 3 minutes, about 4 minutes, or about 5 minutes.

In some intravenous embodiments, the heparinoid is administered by continuous intravenous infusion.

In some embodiments, the non-anticoagulating, non-LMWH, heparinoid is administered as one or more bolus intravenous injections preceded and/or followed by continuous intravenous infusion.

In other embodiments, the heparinoid is administered by subcutaneous injection.

### 4.2.2. Effective Amounts

The non-anticoagulating, non-LMWH heparinoid is administered in amounts effective to achieve the results respectively desired for the methods described above.

In some embodiments, the non-anticoagulating, non-LMWH, heparinoid is administered as an intravenous infusion.

In typical embodiments, the non-anticoagulating, non-LMWH heparinoid is administered by intravenous infusion at a dose rate that maintains the patient's aPTT in the high normal range. In certain embodiments, the non-anticoagulating, non-LMWH heparinoid is ODSH, and is infused at a rate of 0.25-0.375 mg/kg/hr.

In certain embodiments, the non-LMWH heparinoid is administered by intravenous infusion at a dose rate that maintains the patient's aPTT above the high normal range, in the range typically used for DVT prophylaxis. In certain embodiments, the non-anticoagulating, non-LMWH heparinoid is ODSH, and is infused at a rate of from 0.375 mg/kg/hr to about 0.5 mg/kg/hr.

In certain embodiments in which ODSH is the non-anticoagulating, non-LMWH heparinoid, the infusion is at a dose rate of at least about 0.1 mg/kg/hr, at least about 0.2 mg/kg/hr, at least about 0.3 mg/kg/hr, at least about 0.4 mg/kg/hr, at least about 0.5 mg/kg/hr. In various embodiments, ODSH is administered at an infusion rate of no more than about 1.0 mg/kg/hr.

### 4.2.3. Duration, Frequency, and Adjunctive Administration

The duration and frequency of administering the non-anticoagulating, non-LMWH, heparinoid can take into account, among others, the effectiveness of the dosing regimen and the patient's symptoms.

In various embodiments, infusions at the above-described dose rates are administered continuously for up to 7 days. In certain embodiments infusions at the above-described dose rates are administered continuously for up to 6 days, 5 days, 4 days, or 3 days. In some embodiments, infusions at the above-described dose rates are administered continuously for up to 2 days or up to 24 hours. In some embodiments, infusions at the above-described rates are administered for up to 4 hours, 5 hours, 6 hours, 7 hours, 8 hours, 12 hours, 16 hours, or up to 24 hours or more.

In some embodiments, the non-anticoagulating, non-LMWH, heparinoid is administered as an initial bolus of about 20 mg/kg, optionally followed by an infusion of up to about 2 mg/kg/hour for at least about 4 hours, up to about 8 hours, up to about 12 hrs, up to about 16 hours, even up to about 24 hours. In one embodiment, the non-anticoagulating, non-LMWH, heparinoid is administered as an initial bolus of about 8 mg/kg, optionally followed by an infusion of about 0.5 mg/kg/hour for at least about 8 hours. In some embodiments, the non-anticoagulating, non-LMWH, heparinoid is administered as an intravenous bolus at a dose of about 4 mg/kg, optionally followed by an intravenous infusion of the heparinoid at a dose of about 0.25 mg/kg/hr-about 0.375 mg/kg/hr for at least 24 hours. In some embodiments, the non-anticoagulating, non-LMWH, heparinoid is administered as an intravenous bolus at a dose of about 4 mg/kg, followed by a continuous intravenous infusion at a rate of 0.25 mg/kg/hr for a total of 7 days.

For subcutaneous administration, non-anticoagulating, non-LMWH, heparinoid can be administered at doses ranging from about 25 mg to about 400 mg, about 50 mg to about 300 mg, or about 75 mg to about 200 mg, in volumes of 2.0 mL or less per injection.

In various embodiments, non-anticoagulating, non-LMWH, heparinoid administration is repeated. For example, in certain embodiments, heparinoid is administered once daily, twice daily, three times daily, four times daily, five times daily, every two days, every three days, every five days, once a week, once every two weeks, once a month, or every other month. In some embodiments, the heparinoid is administered at regular intervals over a period of several days or weeks, followed by a period of rest, during which no heparinoid is administered. For example, in some embodiments, non-anticoagulating, non-LMWH, heparinoid is administered for one, two, three, or more days, followed by one, two, three, or more days without heparinoid administration. In another exemplary embodiment, the non-anticoagulating, non-LMWH, heparinoid is administered for one, two, three, or more weeks, followed by one, two, three, or more weeks without heparinoid administration. The repeated administration can be at the same dose or at a different dose. The non-anticoagulating, non-LMWH, heparinoid can be administered in one or more bolus injections, one or more infusions, or one or more bolus injections followed and/or preceded by infusion.

The frequency of dosing can be based on and adjusted for the pharmacokinetic parameters of the non-anticoagulating, non-LMWH, heparinoid, the route of administration, and the desired physiological and/or therapeutic effect. Dosages are adjusted to provide sufficient levels of the heparinoid or to maintain the desired physiological effect and/or a therapeutic effect. Any effective administration regimen regulating the timing and sequence of doses may be used, as discussed herein.

Accordingly, the pharmaceutical compositions can be administered in a single dose, multiple discrete doses, continuous infusion, sustained release depots, or combinations thereof, as required to maintain desired minimum level of the agent. Daily dosages may vary, depending on the specific activity of the particular heparinoid. Depending on the route of administration, a suitable dose may be calculated according to, among others, body weight, body surface area, or organ size. The final dosage regimen will be determined by the attending physician in view of good medical practice, considering various factors that modify the action of drugs, e.g., the agent's specific activity, the responsiveness of the patient, the age, condition, body weight, sex, and the like. Additional factors that may be taken into account include time and frequency of administration, drug combination(s), reaction sensitivities, and tolerance/response to therapy. Further refinement of the dosage appropriate for method involving any of the formulations mentioned herein is done by the skilled practitioner, especially in light of the dosage information and assays disclosed, as well as the pharmacokinetic data observed in clinical trials. The amount and/or frequency of the dosage can be altered, increased, or reduced, depending on the subject's response and in accordance with standard clinical practice. The proper dosage and treatment regimen can be established by monitoring the progress of therapy using conventional techniques known to skilled artisans. Appropriate dosages may be ascertained through use of established assays for determining concentration of the heparinoid in a body fluid or other sample together with dose response data.

In embodiments in which non-anticoagulating, non-LMWH, heparinoid is administered to a subject in combination with other therapeutic agents, the heparinoid is administered in a physiologically and/or therapeutically effective temporal proximity to the treatment regimen with the other therapeutic. Administration of a non-anticoagulating, non-LMWH, heparinoid can be concurrent with (at the same time), sequential to (at a different time but on the same day, e.g., during the same patient visit), or separate from (on a different day) the treatment with the other therapeutic. In some embodiments, the non-anticoagulating, non-LMWH, heparinoid is administered concurrently, sequentially, and/or separately from the other agent or therapy being administered. When administered sequentially or separately, the non-anticoagulating, non-LMWH, heparinoid can be administered before, after, or both before and after the other treatment.

In embodiments in which the non-anticoagulating, non-LMWH, heparinoid is administered in combination with treatment with another therapeutic agent, the heparinoid can be administrated via the same or different route as the other therapeutic administered in temporal proximity. In some embodiments, the non-anticoagulating, non-LMWH, heparinoid is administered concurrently or sequentially by the same route. For example, in some embodiments, the non-anticoagulating, non-LMWH, heparinoid and the other therapeutic are administered intravenously, either concurrently or sequentially. Optionally, as part of a treatment regimen, the non-anticoagulating, non-LMWH, heparinoid can further be administered separately (on a different day) from the other therapeutic by a different route, e.g., subcutaneously. In some embodiments, the non-anticoagulating, non-LMWH, heparinoid is administered intravenously on the same day, either at the same time (concurrently), a different time (sequentially), or both concurrently and sequentially with the other therapeutic, and is also administered subcutaneously on one or more days when the patient is not receiving other treatment. In some embodiments, the non-anticoagulating, non-LMWH, heparinoid is administered concurrently or sequentially by a different route. Optionally, as part of a treatment regimen, the non-anticoagulating, non-LMWH, heparinoid can further be administered separately (on a different day) from the other therapeutic by the same or different route as that by which the other therapeutic is administered.

### 4.3. Pharmaceutical Compositions and Unit Dosage Forms

In the methods described herein, the non-anticoagulating, non-LMWH, heparinoid is administered in the form of a pharmaceutical composition.

In typical embodiments, the pharmaceutical composition comprises the non-anticoagulating, non-LMWH, heparinoid and a pharmaceutically acceptable carrier, excipient, and/or diluent, and is formulated for parenteral administration.

### 4.3.1. Pharmaceutical Compositions Formulated for I.V. Administration

In certain embodiments, pharmaceutical compositions of the heparinoid are formulated in volumes and concentrations suitable for intravenous administration. In some embodiments, the composition is formulated for bolus administration. In certain embodiments, pharmaceutical compositions of the heparinoid are formulated in volumes and concentrations suitable for intravenous infusion.

Typical embodiments formulated for intravenous administration comprise the heparinoid in concentrations of at least about 10 mg/ml. In various embodiments, the heparinoid is present in a concentration of at least about 15 mg/ml, at least about 20 mg/ml, at least about 30 mg/ml, at least about 40 mg/ml, at least about 50 mg/ml. In certain embodiments, the heparinoid is packaged in sterile-filled 10 ml glass vials containing an isotonic 50 mg/ml solution of heparinoid in buffered saline.

### 4.3.2. Pharmaceutical Compositions Formulated for S.C. Administration

In various embodiments, the pharmaceutical composition is formulated for subcutaneous administration.

In certain such embodiments, the non-anticoagulating, non-LMWH, heparinoid is associated with multivalent cations. The term “associated”, when used to describe the relationship between a heparinoid and a cation, means a chemically relevant association. The association may be as a salt, ion/counterion, complex, binding, coordination or any other chemically relevant association. The exact nature of the association will be readily apparent to a person of skill in the art depending on the form of the composition.

In various such embodiments, the multivalent cations are selected from cations having a charge of +2, +3, +4, or greater. In some embodiments, the multivalent cation is an ion that contains both positive and negative charges, with a net charge greater than +1. Exemplary multivalent cations include metal ions, amino acids, and other organic and inorganic cations. In certain embodiments, the ion is a metal ion that is Zn²⁺, Ca²⁺, Mg²⁺ or Fe²⁺. In a specific embodiment, the cation is Ca²⁺. In another specific embodiment, the cation is Mg²⁺.

In certain of the embodiments of pharmaceutical composition intended for subcutaneous administration, the heparinoid is associated primarily with one species of multivalent cation. In other embodiments, the heparinoid is associated with several different multivalent cation species. In specific embodiments, the heparinoid is associated with Mg²⁺ and Ca²⁺.

In the multivalent cation embodiments, multivalent cations may be introduced to the heparinoid composition at any step.

In one embodiment, the heparinoid is substantially desulfated at the 2-O and 3-O positions, and the multivalent cation is present during alkaline hydrolysis of the heparin starting material. In certain embodiments, the multivalent cation is present as the chloride salt. In certain embodiments, the multivalent cation is present as the hydroxide salt. In one embodiment, the chloride salt is preferred for use during solution phase alkaline hydrolysis. In another embodiment, the hydroxide salt is preferred for use during solid phase alkaline hydrolysis. In another embodiment, the hydroxide salt is preferred for use when alkaline hydrolysis is performed as a paste. Certain multivalent cations may affect the level of desulfation if present during alkaline hydrolysis, and may be used to achieve desired levels of desulfation. The amount of the multivalent cation may be titrated to control the amount of desulfation as described in U.S. Pat. No. 5,296,471 at Example 4 therein.

Thus, when a multivalent cation is used during alkaline hydrolysis, the multivalent cation concentration used should be adjusted based on both the desired level of desulfation and the desired concentration of the final product. The molar multivalent cation concentration used during alkaline hydrolysis may be substantially less than the molar heparin concentration. Preferably, the molar ratio (multivalent cation:heparin) is about 00.1, 0.2, 0.3, 0.4, 0.5, 0.6, 0.7, 0.8, 0.9, 1.0, 1.1, 1.2, 1.3, 1.4, or 1.5, or any ranges composed of those values. Preferably, the concentration of the multivalent cation used during alkaline hydrolysis is about 0.01 mM, 0.05 mM, 0.1 mM, 0.5 mM, 1 mM, 5 mM, 10 mM, 20 mM, 50 mM, 100 mM, 250 mM, 500 mM or 1 M or any range composed of those numbers.

In certain embodiments, primarily monovalent cations are present during the cold alkaline hydrolysis step, and the multivalent cation is added later, during reconstitution of the lyophilate. In a most preferred embodiment, either MgCl₂ or CaCl₂ is added at high concentration during reconstitution of the lyophilate.

The multivalent cation concentration used during reconstitution may be equal to the concentration of the cation used during alkaline hydrolysis. Preferably, the multivalent cation concentration is at least about 2-fold, 3-fold, 4-fold, 5-fold, 6-fold, 7-fold, 8-fold, 9-fold, 10-fold, 15-fold, 20-fold, 25-fold, 30-fold, 50-fold, 75-fold, 100-fold, 150-fold, 200-fold, 250-fold, 500-fold, or 1000-fold the concentration of the cation used during alkaline hydrolysis. Preferably, the concentration of the multivalent cation used during reconstitution is about 0.1 M, 0.5 M, 1 M, 2 M, 3 M, 4 M, 5M, or greater. Most preferably, the concentration is about 2 M.

Excess cations can be removed by any method known to those in the art. One preferred method of removing excess cations is the use of a desalting column. Another preferred method of removing excess cations is dialysis. After removal of excess ions, the solution preferably has about equal, 2-fold, 3-fold, 4-fold, 5-fold, 6-fold, 7-fold, 8-fold, 9-fold, 10-fold, 15-fold, 20-fold, 25-fold, 30-fold, 50-fold, 75-fold, 100-fold, 150-fold, 200-fold, 250-fold, 500-fold, or 1000-fold greater multivalent cation concentration to monovalent cation concentration. The solution may also be free or substantially free of monovalent cations.

In typical embodiments, the final concentration of non-anticoagulating, non-LMWH, heparinoid in the pharmaceutical composition is between 0.1 mg/mL and 600 mg/mL. In certain embodiments, the final concentration of partially desulfated heparin in the pharmaceutical composition is between 200 mg/mL and 400 mg/mL.

In some embodiments, the concentration of heparinoid is greater than about 25 mg/mL. In certain embodiments, the concentration of heparinoid is greater than about 50 mg/mL. In a variety of embodiments, the concentration of heparinoid is greater than about 60 mg/mL, 70 mg/mL, 80 mg/mL, 90 mg/mL, or 100 mg/mL.

In specific embodiments, the heparinoid is present in the pharmaceutical composition in a concentration greater than about 110 mg/mL, 120 mg/mL, 130 mg/mL, 140 mg/mL, 150 mg/mL, 160 mg/mL, 170 mg/mL, 180 mg/mL, or even greater than about 190 mg/mL or 200 mg/mL. In specific embodiments, the heparinoid is present in the pharmaceutical composition at a concentration of about 175 mg/mL. In another embodiment, the heparinoid is present in the pharmaceutical composition at a concentration of about 200 mg/mL. In one embodiment, the heparinoid is present in the pharmaceutical composition at a concentration of 400 mg/mL.

In certain embodiments, the concentration of non-anticoagulating, non-LMWH, heparinoid is 50 mg/mL to 500 mg/mL, 100 mg/mL to 400 mg/mL, or 150 mg/mL to 300 mg/mL. In specific embodiments, the concentration is 50 mg/mL, 100 mg/mL, 150 mg/mL, 200 mg/mL, 250 mg/mL, 300 mg/mL, 350 mg/mL, 400 mg/mL, 450 mg/mL or 500 mg/mL. In certain currently preferred embodiments, the concentration is 200 mg/mL, 300 mg/mL or 400 mg/mL.

In typical embodiments, the pharmaceutical composition has a viscosity of less than about 100 cP. In various embodiments, the pharmaceutical composition has a viscosity of less than about 80 cP. In certain embodiments, the pharmaceutical composition has a viscosity of less than about 60 cP. In particular embodiments, the pharmaceutical composition has a viscosity of less than about 20 cP.

In typical embodiments, the pharmaceutical composition has an osmolality less than about 2500 mOsm/kg. In various embodiments, the pharmaceutical composition has an osmolality between about 150 mOsm/kg and about 500 mOsm/kg. In certain embodiments, the pharmaceutical composition has an osmolality between about 275 mOsm/kg and about 300 mOsm/kg. In a particular embodiment, the pharmaceutical composition has an osmolality of about 285 mOsm/kg. In a specific embodiment, the pharmaceutical composition is isotonic.

### 5. EXAMPLES

Practice of the various embodiments of the methods can be understood through reference to the following examples, which are provided by way of,l illustration and are not intended to be limiting.

### 5.1. Example 1

### ODSH Reduces Secondary Brain Injury After Middle Cerebral Artery Occlusion In an Animal Model

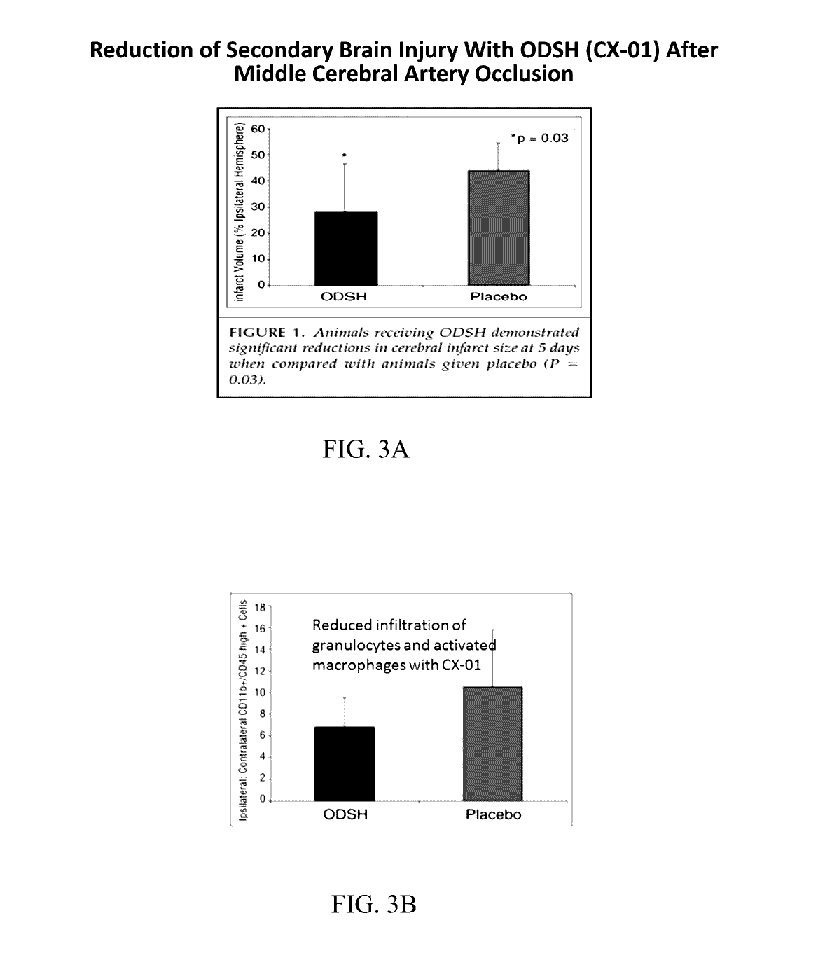

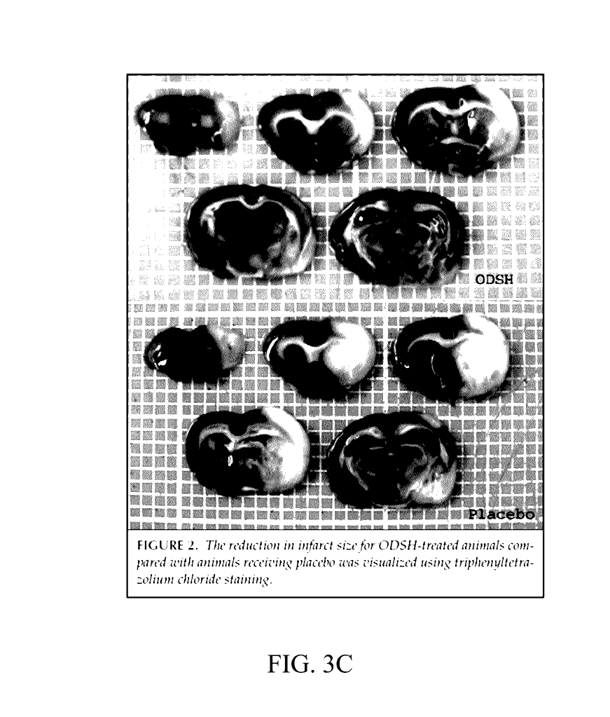

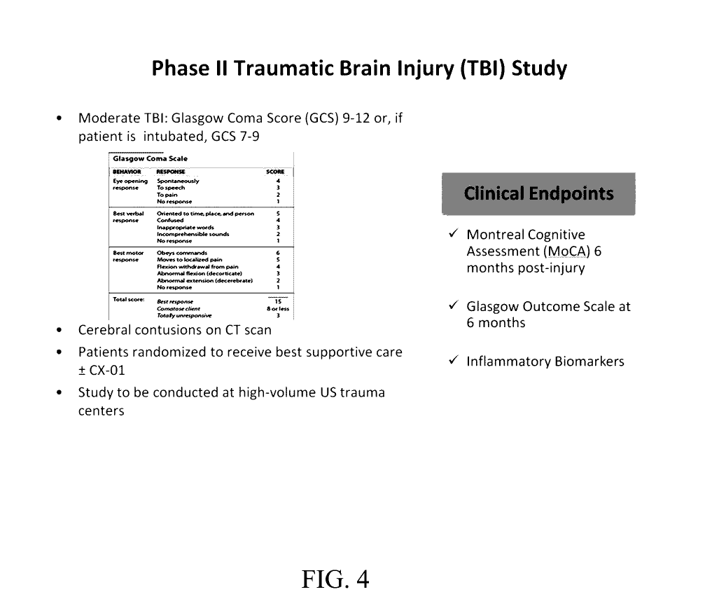

As shown in FIGS. 3A-3C, ODSH reduced secondary brain injury after cerebral artery occlusion in an animal model.

### 5.2. Example 2

### Clinical Trial

A clinical trial is conducted.

Enrollment criteria are (i) moderate TBI: Glasgow Coma Score (GCS) of 9-12, or if patient is intubated, GCS 7-9; (ii) cerebral contusions on CT scan. Patients are randomized into 2 groups. Both groups receive best supportive care. This is an active treatment control. One group additionally receives infusion of ODSH.

Results.

After unblinding, it is found that adding ODSH infusion to best supportive care improves Montreal Cognitive Assessment (MoCA) score six months post-injury, Glasgow Outcome Scale scores at 6 months, and reduces inflammatory biomarkers.

All publications, patents, patent applications and other documents cited in this application are hereby incorporated by reference in their entireties for all purposes to the same extent as if each individual publication, patent, patent application or other document were individually indicated to be incorporated by reference for all purposes.

While various specific embodiments have been illustrated and described, it will be appreciated that various changes can be made without departing from the spirit and scope of the invention(s).

What is claimed is:

1. A method for treating a subject suffering from a disorder selected from the group consisting of traumatic brain injury (TBI), traumatic spine injury, lung injury associated with TBI, acute respiratory distress syndrome (ARDS), intracerebral hemorrhage, subarachnoid hemorrhage, comprising: administering to the subject an effective amount of non-anticoagulating, non-LMWH heparinoid.

2. The method of claim 1, wherein the disorder is TBI.

3. The method of claim 1, wherein the disorder is lung injury associated with TBI.

4. The method of claim 3, wherein the lungs are subsequently transplanted.

5. The method of claim 1, wherein the heparinoid is substantially desulfated at the 2-O position and/or 3-O position.

6. The method of claim 5, wherein the 2-O and/or 3-O-desulfated heparin derivative is not substantially desulfated at the 6-O or N positions.

7. The method of claim 5, wherein the heparinoid is at least 85% desulfated at the 2-O position.

8. The method of claim 5, wherein the heparinoid is at least 85% desulfated at the 3-O position.

9. The method of claim 5, wherein the heparinoid is at least 85% desulfated at each of the 2-O and 3-O positions.

10. The method of claim 5, wherein the heparinoid is at least 95% desulfated at each of 2-O and 3-O positions.

11. The method of claim 5, wherein the heparinoid has an average molecular weight of about 8 kDa to about 15 kDa.

12. The method of claim 5, wherein the heparinoid has an average molecular weight of about 11 kDa to about 13 kDa.

13. The method of claim 5, wherein the non-anticoagulating, non-LMWH, heparinoid is obtained by alkaline hydrolysis of unfractionated heparin.

14. The method of claim 5, wherein the non-anticoagulating, non-LMWH, heparinoid is associated with a multivalent cation.

15. The method of claim 14, wherein the multivalent cation is Mg²⁺ or Ca²⁺.

16. The method of claim 1, wherein the heparinoid is administered intravenously.

17. The method of claim 16, wherein the heparinoid is administered as a continuous infusion.

18. The method of claim 17, wherein the heparinoid is administered by a continuous intravenous infusion, wherein the infusion rate is titrated to the high normal range for aPTT.

19. The method of claim 17, wherein the ODSH is administered by a continuous intravenous infusion, wherein the infusion rate is titrated to above the high normal range for aPTT.

20. The method of claim 17, wherein the heparinoid is ODSH.

21. The method of claim 18, wherein the ODSH is administered as an intravenous infusion of 0.25 mg/kg/hr-0.375 mg/kg/hr.

22. The method of claim 19, wherein the ODSH is administered as an intravenous infusion of 0.375 mg/kg/hr-0.5 mg/kg/hr ODSH.

23. The method of claim 1, wherein the TBI is selected from the group consisting of:
open head injury, closed head injury, concussion, contusion. Diffuse Axonal Injury, Coup-contre coup injury, Second Impact Syndrome, penetrating injury, Shaken Baby Syndrome, Locked in Syndrome, anoxic brain injury and hypoxic brain injury.

24. The method of claim 1, wherein the treatment results in reduced cerebral edema, reduced cerebral hypoxia, reduced cerebral ischemia, improved cognitive ability or increased survival in the subject compared to a subject suffering from the disorder and who has not been treated with the non-anticoagulating, non-LMWH heparinoid.

25. The method of claim 1, wherein the treatment results in reduction of lung dysfunction in the subject compared to a subject suffering from the disorder and who has not been treated with non-anticoagulating, non-LMWH heparinoid, and wherein the lung dysfunction is selected from the group consisting of alveolar hemorrhage, pulmonary vascular leakage, neutrophil infiltration and ARDS.

26. The method of claim 25, wherein the lung dysfunction is exacerbated by mechanical ventilation.

27. The method of claim 1, wherein the treatment results in reduced pulmonary vascular pressure, reduced neurogenic pulmonary edema, reduced pulmonary inflammation or increased survival in the subject compared to a subject suffering from the disorder and who has not been treated with non-anticoagulating, non-LMWH heparinoid.

28. A method for treating a subject prior to or following lung transplantation comprising administering to the patient an effective amount of non-anticoagulating, non-LMWH heparinoid.

29. The method of claim 28, wherein a donor lung used for the transplantation had been taken from a subject that had incurred a TBI.

30. The method of claim 28, wherein the treatment results in reduced lung ischemia-reperfusion injury or increased survival compared to a subject not treated with non-anticoagulating, non-LMWH heparinoid.

31. A method of preparing an isolated lung for transplantation ex-vivo, comprising contacting the lung with non-anticoagulating, non-LMWH heparinoid.

32. The method of claim 31, wherein the lung has been isolated from a subject that has incurred a TBI.

33. The method of claim 31, wherein the preparation results in reduced lung ischemia-reperfusion injury or increased survival in a subject that is the recipient of the lung following transplantation as compared to a subject following lung transplantation that is not a recipient of a lung that has been contacted with non-anticoagulating, non-LMWH heparinoid.

34. The method of claim 31, wherein the lung is perfused with a solution comprising the non-anticoagulating, non-LMWH heparinoid.
